(19) United States
(12) Patent Application Publication
(10) Pub. No.: US 2006/0117396 A1
(43) Pub. Date: Jun. 1, 2006

(54) Assay methods for identifying modulators of OPSDL3 and transgenic knockouts

(76) Inventors: Andrew J. Murphy, Croton-on-Hudson, NY (US); Y. Gopi Shanker, New York, NY (US); Susan Croll-Kalish, Tarrytown, NY (US); Richard Torres, New York, NY (US)

Correspondence Address: REGENERON PHARMACEUTICALS, INC, 777 OLD SAW MILL RIVER ROAD, TARRYTOWN, NY, 10591, US

(21) Appl. No.: 11/292,150

(22) Filed: Nov. 30, 2005

Related U.S. Application Data
(60) Provisional application No. 60/631,827, filed on Nov. 30, 2004.

(51) Int. Cl. A01K 67/00; G01N 33/53

(52) U.S. Cl. 800/8; 435/7.1

(57) ABSTRACT

Methods for treating or alleviating pain by treating a subject in need thereof with an agonist of OPSDL3 activity or expression. Further provided are screening methods for identifying agents capable of activating OPSDL3, and transgenic non-human animals which homozygous for deletion of the endogenous OPSDL3 gene and/or comprise a human OPSDL3 gene.

### CROSS-REFERENCE TO RELATED APPLICATIONS

This application claims the benefit under 35 USC § 119(e) of U.S. Provisional application 60/631,827 filed 30 Nov. 2004, which application is herein specifically incorporated by reference in its entirety.

### BACKGROUND OF THE INVENTION

1. Field of the Invention

This invention is related to methods of using G-protein-coupled receptor (GPCR) nucleic acids and polypeptides. More specifically, the invention relates to assay methods for identifying an agonist of OPSDL3, and therapeutic methods of treating pain with such agonists.

2. Description of Related Art

OPSDL3 or rhodopsin-like 3 is an orphan receptor initially identified in the rat as a G-protein coupled receptor, and named “leda” (induced early in differentiating astrocytes) (De Smet et al. (2002) J. Neurochem. 81(3):575-0660). In adult rats, expression was found exclusively in the brain and testis.

### BRIEF SUMMARY OF THE INVENTION

The experiments described below reveal, for the first time, the functional role of OPSDL3 in pain transmission. These results allow the development of screening methods for the development of a class of potent non-opiate analgesics unencumbered by one or more of the undesirable side effects of opiates or opiate-receptor ligands, and therapeutic methods for treatment of pain.

Accordingly, in a first aspect, the invention provides methods for screening for agents capable of binding a human OPSDL3 protein. More specifically, the invention provides methods of identifying agents capable of modulating (e.g., enhancing or inhibiting) human OPSDL3-mediated activity. The screening methods of the invention include in vitro and in vivo assays. Agents capable of modulating OPSDL3-mediated activity preferably include agents capable of activating OPSDL3 modulation of pain sensation.

In a second aspect, the invention features a method of alleviating or inhibiting a OPSDL3-mediated condition, comprising administering an agent capable of enhancing OPSDL3 activity or expression. In specific embodiments, the agent is a peptide, a small molecule, an activating antibody or fragment thereof. In one embodiment, the condition being alleviated is pain. In one embodiment, the pain being treated may be a neuropathic pain disease. In another embodiment, the pain condition results from an injury to the body, including surgery, medical treatment, or accident. In another embodiment, the pain condition is related to childbirth. In another embodiment, the therapeutic method of the invention comprising administering an agent capable of activating OPSDL3 with a second pain-relieving agent, e.g., an opiate or an opioid. In this embodiment, the therapeutic method may allow a decreased amount of the second agent to be administered when administered in combination with an agent of the invention.

In a third aspect, the invention features a method of reducing the amount of a first agent required to achieve a desired level of analgesia, by administering with the first agent, a second agent, wherein the second agent is an agent capable of activating OPSDL3. In one embodiment, the first agent is an opiate and the second agent is an activating antibody or fragment thereof. In a more specific embodiment, the opiate is morphine.

In a fourth aspect, the invention features pharmaceutical compositions useful for treatment of pain or nociception comprising an agent capable of activating OPSDL3 activity or expression. In one embodiment, the agent is identified by a screening method of the invention.

In a fifth aspect, the invention features a transgenic animal comprising a modification of an endogenous OPSDL3 gene. As described more fully in U.S. Pat. No. 6,586,251, the transgenic animal of the invention is generated by targeting the endogenous OPSDL3 gene with a large targeting vector (LTVEC). In one embodiment of the transgenic animal of the invention, the animal is a knock-out wherein the OPSDL3 gene is altered or deleted such that the function of the endogenous OPSDL3 protein is reduced or ablated. In another embodiment, the transgenic animal is a knock-in animal modified to comprise an exogenous gene. In a more specific embodiment of the knock-in transgenic animal of the invention, the transgene is a human OPSDL3 gene. Such transgenic animals are useful, for example, in identifying agents specifically inhibiting pain or sensation mediated by the human OPSDL3 protein.

Other objects and advantages will become apparent from a review of the ensuing detailed description.

### BRIEF DESCRIPTION OF THE FIGURES

FIG. 1 is a bar graph of the results of a hot plate test of wild-type (n=12) and OPSDL3 knock-out (n=10) mice.

FIG. 2 is a bar graph of the results of a tail flick test of wild-type (n=12) and OPSDL3 knock-out (n=10) mice. OPSDL3 knock-out mice had a significantly (p<0.0025) decreased tail flick latency.

FIG. 3. Transfection of hOPSD-like3 caused an appreciable increase in the CRE-luciferase activity suggesting that activation of hOPSDL3 leads to an increase in intracellular cyclic-AMP (cAMP).

### DETAILED DESCRIPTION

Before the present methods are described, it is to be understood that this invention is not limited to particular methods, and experimental conditions described, as such methods and conditions may vary. It is also to be understood that the terminology used herein is for the purpose of describing particular embodiments only, and is not intended to be limiting, since the scope of the present invention will be limited only by the appended claims.

As used in this specification and the appended claims, the singular forms “a”, “an”, and “the” include plural references unless the context clearly dictates otherwise. Thus for example, references to “a method” includes one or more methods, and/or steps of the type described herein and/or which will become apparent to those persons skilled in the art upon reading this disclosure and so forth.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference.

Definitions

By the term “OPSDL3-associated” or “OPSDL3-mediated” condition or disease is meant a condition which is affected directly or indirectly by modulation of OPSDL3 activity. As established in the experiments described below, an OPSDL3-mediated condition is pain transmission which can be inhibited with an agent capable of binding and modulating OPSDL3.

General Description

This invention is based in part on elucidation of the function of the OPSDL3 as involved in the modulation of nociception, pain, and/or thermal sensation. Accordingly, these discoveries provide new methods for the treatment of pain and OPSDL3-mediated conditions.

Therapeutic Methods and Combination Therapies

The invention is directed to therapeutically useful methods for treating any disease or condition which is improved, ameliorated, inhibited or prevented by modulation of OPSDL3. Generally, activation of OPSDL3 results in an analgesic effect, e.g., alleviation of pain or discomfort caused by pain. Modulation of OPSDL3 may be desirable in a number of situations, including for example, to alleviate pain associated with neuropathy, labor and childbirth, and/or injury. In numerous embodiments, a modulator of OPSDL3 may be administered in combination with one or more additional compounds or therapies. For example, an antibody capable of binding and activating OPSDL3, or a biologically active fragment thereof, may be co-administered, or administered in conjunction with one or more therapeutic compounds.

Screening Assays

The present invention provides methods for identifying agents (e.g., candidate compounds or test compounds) that are capable of modulating (e.g., upregulating, activating, enhancing) human rhodopsin-like 3-mediated activity. Preferably, the invention provides methods for identifying agents capable of effecting OPSDL3 modulation of nociception or pain. Agents identified through the screening method of the invention are potential therapeutics for use in providing pain relief to a subject in need thereof. Examples of agents include, but are not limited to, nucleic acids (e.g., DNA and RNA), carbohydrates, lipids, proteins, peptides, peptidomimetics, small molecules, antibodies, antibody fragments, and other drugs. Agents can be obtained using any of the numerous approaches in combinatorial library methods known in the art.

In one embodiment, agents that bind OPSDL3 are identified in a cell-based assay system. In accordance with this embodiment, cells expressing a OPSDL3 protein or protein fragment are contacted with a candidate (or a control compound), and the ability of the candidate compound to bind OPSDL3 is determined. The cell may be of prokaryotic origin (e.g., E. coli) or eukaryotic origin (e.g., yeast or mammalian). In specific embodiments, the cell is a OPSDL3 expressing mammalian cell, such as, for example, a COS-7 cell, a 293 human embryonic kidney cell, a NIH 3T3 cell, or Chinese hamster ovary (CHO) cell. Further, the cells may express a OPSDL3 protein or protein fragment endogenously or be genetically engineered to express a OPSDL3 protein or protein fragment. In some embodiments of the binding assays of the invention, the compound to be tested may be labeled. Cells expressing the OPSDL3 are then incubated with labeled test compounds, in binding buffer, in cell culture dishes. To determine non-specific binding, unlabeled ligand may be added to the wells. After the incubation, bound and free ligands are separated and detection activity measured in each well.

The ability of the candidate compound to alter the activity of OPSDL3 can be determined by methods known to those of skill in the art, for example, by flow cytometry, a scintillation assay, immunoprecipitation or western blot analysis. For example, modulators of OPSDL3 activity may be identified using a biological readout in cells expressing an OPSDL3 protein or protein fragment. Agonists or antagonists are identified by incubating cells or cell fragments expressing OPSDL3 with test compound and measuring a biological response in these cells and in parallel cells or cell fragments not expressing OPSDL3. An increased biological response in the cells or cell fragments expressing OPSDL3 compared to the parallel cells or cell fragments indicates the presence of an agonist in the test sample, whereas a decreased biological response indicates an antagonist.

In more specific embodiments, detection of binding and/or modulation of a test agent to a OPSDL3 protein may be accomplished by detecting a biological response, such as, for example, measuring Ca²⁺ ion flux, cAMP, IP₃, PIP₃ and transcription of reporter genes. For example, to identify ligands of OPSDL3, cells expressing the receptor may be screened against a panel of know compounds utilizing a bioluminescent signal such as the aequorin luminescence assays (see, for example, Button et al. (1993) Cell. Calcium 14:663-671; Liu et al. (1999) Biochem. Biophys. Res. Comm. 266:174-178; Ungrin et al. (1999) Anal. Biochem. 272:34-42; Fujii et al. (2000) J. Biol. Chem 275:21086-21074; Raddatz et al. (2000) J. Biol. Chem. 275:32452-32459; and Shan et al. (2000) J. Biol. Chem. 275:39482-39486, which references are herein specifically incorporated by reference in their entireties). Suitable reporter genes include endogenous genes as well as exogenous genes that are introduced into a cell by any of the standard methods familiar to the skilled artisan, such as transfection, electroporation, lipofection and viral infection. The invention further includes other end point assays to identify compounds that modulate (stimulate or inhibit) receptor activity, such as those associated with signal transduction.

In another embodiment, agents that modulate OPSDL3-mediated activity are identified in a cell-free assay system. In accordance with this embodiment, an OPSDL3 protein or protein fragment is contacted with a test (or control) compound and the ability of the test compound to bind OPSDL3 is determined. Competitive binding may also be determined in the presence of an OPSDL3 ligand. In vitro binding assays employ a mixture of components including an OPSDL3 protein or protein fragment, which may be part of a fusion product with another peptide or polypeptide, e.g., a tag for detection or anchoring, and a sample suspected of containing a natural OPSDL3 binding target. A variety of other reagents such as salts, buffers, neutral proteins, e.g., albumin, detergents, protease inhibitors, nuclease inhibitors, and antimicrobial agents, may also be included. The mixture components can be added in any order that provides for the requisite bindings and incubations may be performed at any temperature which facilitates optimal binding. The mixture is incubated under conditions whereby the OPSDL3 protein binds the test compound. Incubation periods are chosen for optimal binding but are also minimized to facilitate rapid, high-throughput screening.

After incubation, the binding between the OPSDL3 protein or protein fragment and the suspected binding target is detected by any convenient way. When a separation step is useful to separate bound from unbound components, separation may be effected by, for example, precipitation or immobilization, followed by washing by, e.g., membrane filtration or gel chromatography. One of the assay components may be labeled which provides for direct detection such as, for example, radioactivity, luminescence, optical or electron density, or indirect detection such as an epitope tag or an enzyme. A variety of methods may be used to detect the label depending on the nature of the label and other assay components, e.g., through optical or electron density, radiative emissions, nonradiative energy transfers, or indirectly detected with antibody conjugates.

It may be desirable to immobilize either the receptor protein, or fragment, or its target molecule to facilitate separation of complexes from uncomplexed forms of one of the proteins, as well as to accommodate automation of the assay. Techniques for immobilizing proteins on matrices can be used in the drug screening assays. In one embodiment, a fusion protein is provided which adds a domain that allows the protein to be bound to a matrix. For example, glutathione-S-transferase fusion proteins can be adsorbed onto glutathione sepharose beads (Sigma Chemical, St. Louis, Mo.) or glutathione derivatized microtitre plates, which are then combined with the cell lysates (e.g., ³⁵S-labeled) and the candidate compound, and the mixture incubated under conditions conducive to complex formation (e.g., at physiological conditions for salt and pH). Following incubation, the beads are washed to remove any unbound label, and the matrix immobilized radiolabel determined directly, or in the supernatant after the complexes are dissociated. Alternatively, the complexes can be dissociated from the matrix, separated by SDS-PAGE, and the level of receptor-binding protein found in the bead fraction quantitated from the gel using standard electrophoretic techniques. For example, either the polypeptide or its target molecule can be immobilized utilizing conjugation of biotin and streptavidin using techniques well known in the art. Alternatively, antibodies reactive with the protein but which do not interfere with binding of the protein to its target molecule can be derivatized to the wells of the plate, and the protein trapped in the wells by antibody conjugation. Preparations of a receptor-binding protein and a candidate compound are incubated in the receptor protein-presenting wells and the amount of complex trapped in the well can be quantitated. Methods for detecting such complexes, in addition to those described above for the GST-immobilized complexes, include immunodetection of complexes using antibodies reactive with the receptor protein target molecule, or which are reactive with receptor protein and compete with the target molecule, as well as enzyme-linked assays which rely on detecting an enzymatic activity associated with the target molecule.

In another embodiment, agents that modulate OPSDL3-mediated activity are identified in an animal model. Examples of suitable animals include, but are not limited to, mice, rats, rabbits, monkeys, guinea pigs, dogs and cats. In accordance with this embodiment, the test compound or a control compound is administered (e.g., orally, rectally or parenterally such as intraperitoneally or intravenously) to a suitable animal and the effect on the OPSDL3-mediated activity is determined. More specifically, this method may be used to identify an agent capable of inhibiting nociception or pain transmission. Examples of assays useful for identifying potential therapeutic agents, e.g., agents capable of modulating OPSDL3-mediated activity, include the tail flick assay described below, hot plate assays, or the capsaicin test.

OPSDL3 Antibodies

The term “antibody” covers, for example, single anti-OPSDL3 monoclonal antibodies, anti-OPSDL3 antibody compositions with polyepitopic specificity, single chain anti-OPSDL3 antibodies, and fragments of anti-OPSDL3 antibodies (see below).

The term “monoclonal antibody” as used herein refers to an antibody obtained from a population of substantially homogeneous antibodies, i.e., the individual antibodies comprising the population are identical except for possible naturally occurring mutations that may be present in minor amounts.

“Antibody fragments” comprise a portion of an intact antibody, preferably the antigen binding or variable region of the intact antibody. Examples of antibody fragments include Fab, Fab′, F(ab′)₂, and Fv fragments; diabodies; linear antibodies (Zapata et al. (1995) Protein Eng. 8(10):1057-1062); single-chain antibody molecules, single variable domain antibodies; and multispecific antibodies formed from antibody fragments.

“Fv” is the minimum antibody fragment, which contains a complete antigen-recognition and -binding site. This region consists of a dimer of one heavy- and one light-chain variable domain in tight, non-covalent association. It is in this configuration that the three CDRs of each variable domain interact to define an antigen-binding site on the surface of the V_{H}-V_{L} dimer. Collectively, the six CDRs confer antigen-binding specificity to the antibody. However, even a single variable domain (or half of an Fv comprising only three CDRs specific for an antigen) has the ability to recognize and bind antigen.

Antibodies exist as intact immunoglobulins, or as a number of well-characterized fragments produced by digestion with various peptidases. For example, papain digestion of antibodies produces two identical antigen-binding fragments, called “Fab” fragments, each with a single antigen-binding site and a residual “Fc” fragment, whose name reflects its ability to crystallize readily. Pepsin digests an antibody below the disulfide linkages in the hinge region to produce F(ab)′₂, a dimer of Fab′ which itself is a light chain joined to V_{H}-C_{H} by a disulfide bond. The F(ab)′₂ may be reduced under mild conditions to break the disulfide linkage in the hinge region, thereby converting the F(ab)′₂ dimer into an Fab′ monomer. The Fab′ monomer is essentially Fab with part of the hinge region. While various antibody fragments are defined in terms of the digestion of an intact antibody, one of skill will appreciate that such fragments may be synthesized de novo either chemically or by using recombinant DNA methodology. Thus, the terms antibody, as used herein, also includes antibody fragments either produced by the modification of whole antibodies, or those synthesized de novo using recombinant DNA methodologies (e.g., single chain Fv) (scFv)) or those identified using phase display libraries (see, for example, McCafferty et al. (1990) Nature 348:552-554). In addition, the OPSDL3-binding component of the antibody or antibody fragments of the invention include the variable regions of the heavy (V_{H}) or the light (V_{L}) chains of immunoglobulins, as well as the OPSDL3-binding portions thereof. Methods for producing such variable regions are described in Reiter, et al. (1999) J. Mol. Biol. 290:685-698.

An “isolated” antibody is one which has been identified and separated and/or recovered from a component of its natural environment. Contaminant components of its natural environment are materials which would interfere with diagnostic or therapeutic uses for the antibody, and may include enzymes, hormones, and other proteinaceous or nonproteinaceous solutes. In preferred embodiments, the antibody will be purified (1) to greater than 95% by weight of antibody as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue or, preferably, silver stain. Isolated antibody includes the antibody in situ within recombinant cells since at least one component of the antibody's natural environment will not be present. Ordinarily, however, isolated antibody will be prepared by at least one purification step.

Methods for preparing antibodies are known to the art. See, for example, Kohler & Milstein (1975) Nature 256:495-497; Harlow & Lane (1988) Antibodies: a Laboratory Manual, Cold Spring Harbor Lab., Cold Spring Harbor, N.Y.). The genes encoding the heavy and light chains of an antibody of interest can be cloned from a cell, e.g., the genes encoding a monoclonal antibody can be cloned from a hybridoma and used to produce a recombinant monoclonal antibody. Gene libraries encoding heavy and light chains of monoclonal antibodies can also be made from hybridoma or plasma cells. Random combinations of the heavy and light chain gene products generate a large pool of antibodies with different antigenic specificity. Techniques for the production of single chain antibodies or recombinant antibodies (U.S. Pat. No. 4,946,778; U.S. Pat. No. 4,816,567) can be adapted to produce antibodies used in the fusion proteins and methods of the instant invention. Also, transgenic mice, or other organisms such as other mammals, may be used to express human or humanized antibodies. Alternatively, phage, yeast, E. coli, or mammalian cell display technologies or E. coli periplasmic expression and/or secretion technologies can be used to identify antibodies, antibody fragments, such as variable domains, ScFv, heteromeric Fab fragments, and whole antibodies that specifically bind to selected antigens. Phage display is of particular value to isolate weakly binding antibodies or fragments thereof from un-immunized animals which, when combined with other weak binders in accordance with the invention described herein, create strongly binding fusion polypeptides.

Screening and selection of preferred immunoglobulins (antibodies) can be conducted by a variety of methods known to the art. Initial screening for the presence of monoclonal antibodies specific to OPSDL3 may be conducted through the use of fluorescent cell sorting, panning, or ELISA-based methods, for example. A secondary screen is preferably conducted to identify and select a desired monoclonal antibody having the capacity to activate OPSDL3. Secondary screening may be conducted with any suitable method known to the art. One preferred method, termed “Biosensor Modification-Assisted Profiling” (“BiaMAP”) is described in US patent publication 2004/101920, herein specifically incorporated by reference in its entirety. BiaMAP allows rapid identification of hybridoma clones producing monoclonal antibodies with desired characteristics. More specifically, monoclonal antibodies are sorted into distinct epitope-related groups based on evaluation of antibody: antigen interactions.

The antibodies useful in the method of the invention are identified as those that activate the activity of OPSDL3 in the appropriate assay (e.g., Shimizugawa et al. (2002) J. Biol. Chem., 277:33742-33748)

The terms “biological activity” and “biologically active” with regard to the OPSDL3 or antibody fragments herein refer to the ability of a molecule to specifically bind to OPSDL3 and activate OPSDL3 mediated responses.

Human and Humanized Antibodies

“Humanized” forms of non-human (e.g., murine) antibodies are chimeric immunoglobulins, immunoglobulin chains or fragments thereof (such as Fv, Fab, Fab′, F(ab′)₂ or other antigen-binding subsequences of antibodies) which contain minimal sequences required for antigen binding derived from non-human immunoglobulin. For the most part, humanized antibodies are human immunoglobulins (recipient antibody) in which residues from a CDR of the recipient are replaced by residues from a CDR of a non-human species (donor antibody) such as mouse, rat or rabbit having the desired specificity, affinity, and capacity. In some instances, Fv framework (FR) residues of the human immunoglobulin are replaced by corresponding non-human residues to maintain the binding properties. Furthermore, humanized antibodies may comprise residues which are found neither in the recipient antibody nor in the imported CDR or framework sequences. These modifications are made to further refine and maximize antibody performance. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin sequence. The humanized antibody optimally also will comprise at least a portion of an immunoglobulin constant region (Fc), typically that of a human immunoglobulin. For further details, see, Jones et al. (1986) Nature 321:522-525; Reichmann et al. (1988) Nature 332:323-329; and Presta (1992) Curr. Op. Struct. Biol. 2:593-596.

Fully human antibodies may be made by any method known to the art. For example, U.S. Pat. No. 6,596,541 describes a method of generating fully human antibodies (see also U.S. Pat. No. 6,794,132). Briefly, initially a transgenic animal such as a mouse is generated that produces hybrid antibodies containing human variable regions (VDJ/VJ) and mouse constant regions. This is accomplished by a direct, in situ replacement of the mouse variable region (VDJ/VJ) genes with their human counterparts. The mouse is then exposed to human OPSDL3, or an immunogenic fragment thereof. The resultant hybrid immunoglobulin loci will undergo the natural process of rearrangements during B-cell development to produce hybrid antibodies having the desired specificity. The antibody of the invention is selected as described above. Subsequently, fully-human antibodies are made by replacing the mouse constant regions with the desired human counterparts. In a preferred embodiment, the mouse background is a knockout of the OPSDL3 gene, which enhances the immune response and increases the repertoire of resulting antibodies.

Methods of Administration

The invention provides methods of treatment comprising administering to a subject an effective amount of an agent of the invention. In a preferred aspect, the agent is substantially purified (e.g., substantially free from substances that limit its effect or produce undesired side-effects). The subject is preferably a human subject.

Various delivery systems are known and can be used to administer an agent of the invention, e.g., encapsulation in liposomes, microparticles, microcapsules, recombinant cells capable of expressing the compound, receptor-mediated endocytosis (see, e.g., Wu and Wu, 1987, J. Biol. Chem. 262:4429-4432), construction of a nucleic acid as part of a retroviral or other vector, etc. Methods of introduction can be enteral or parenteral and include but are not limited to intradermal, intramuscular, intraperitoneal, intravenous, subcutaneous, intranasal, epidural, intrathecal, and oral routes. The compounds may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. In addition, it may be desirable to introduce the pharmaceutical compositions of the invention into the central nervous system by any suitable route, including intraventricular and intrathecal injection; intraventricular or intrathecal injection may be facilitated by an intraventricular catheter, for example, attached to a reservoir, such as an Ommaya reservoir. Pulmonary administration can also be employed, e.g., by use of an inhaler or nebulizer, and formulation with an aerosolizing agent.

In a specific embodiment, it may be desirable to administer the pharmaceutical compositions of the invention locally to the area in need of treatment; this may be achieved, for example, and not by way of limitation, by local infusion during surgery, topical application, e.g., by injection, by means of a catheter, or by means of an implant, said implant being of a porous, non-porous, or gelatinous material, including membranes, such as sialastic membranes, fibers, or commercial skin substitutes.

In another embodiment, the active agent can be delivered in a vesicle, in particular a liposome (see Langer (1990) Science 249:1527-1533). In yet another embodiment, the active agent can be delivered in a controlled release system. In one embodiment, a pump may be used (see Langer (1990) supra). In another embodiment, polymeric materials can be used (see Howard et al. (1989) J. Neurosurg. 71:105). In another embodiment where the active agent of the invention is a nucleic acid encoding a protein, the nucleic acid can be administered in vivo to promote expression of its encoded protein, by constructing it as part of an appropriate nucleic acid expression vector and administering it so that it becomes intracellular, e.g., by use of a retroviral vector (see, for example, U.S. Pat. No. 4,980,286), or by direct injection, or by use of microparticle bombardment (e.g., a gene gun; Biolistic, Dupont), or coating with lipids or cell-surface receptors or transfecting agents, or by administering it in linkage to a homeobox-like peptide which is known to enter the nucleus (see e.g., Joliot et al., 1991, Proc. Natl. Acad. Sci. USA 88:1864-1868), etc. Alternatively, a nucleic acid can be introduced intracellularly and incorporated within host cell DNA for expression, by homologous recombination.

Pharmaceutical Compositions

The present invention also provides pharmaceutical compositions. Such compositions comprise a therapeutically effective amount of an active agent, and a pharmaceutically acceptable carrier. In a specific embodiment, the composition comprises a combination of an agent of the invention and a second pain-relieving agent. The term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin.

In a preferred embodiment, the composition is formulated in accordance with routine procedures as a pharmaceutical composition adapted for intravenous administration to human beings. Where necessary, the composition may also include a solubilizing agent and a local anesthetic such as lidocaine to ease pain at the site of the injection. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The active agents of the invention can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

The amount of the active agent of the invention which will be effective in the treatment of a OPSDL3-mediated condition can be determined by standard clinical techniques based on the present description. In addition, in vitro assays may optionally be employed to help identify optimal dosage ranges. The precise dose to be employed in the formulation will also depend on the route of administration, and the seriousness of the condition, and should be decided according to the judgment of the practitioner and each subject's circumstances. However, suitable dosage ranges for intravenous administration are generally about 20-500 micrograms of active compound per kilogram body weight. Suitable dosage ranges for intranasal administration are generally about 0.01 pg/kg body weight to 1 mg/kg body weight. Effective doses may be extrapolated from dose-response curves derived from in vitro or animal model test systems.

Kits

The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects (a) approval by the agency of manufacture, use or sale for human administration, (b) directions for use, or both.

Transgenic Animals

The invention includes a knock-out or knock-in animal having a modified endogenous OPSDL3 gene. The invention contemplates a transgenic animal having an exogenous OPSDL3 gene generated by introduction of any OPSDL3-encoding nucleotide sequence which can be introduced as a transgene into the genome of a non-human animal. Any of the regulatory or other sequences useful in expression vectors can form part of the transgenic sequence. A tissue-specific regulatory sequence(s) can be operably linked to the transgene to direct expression of the OPSDL3 protein to particular cells.

Knock-out animals containing a modified OPSDL3 gene as described herein are useful to identify OPSDL3 function. Methods for generating knock-out or knock-in animals by homologous recombination in ES cells are known to the art. Animals generated from ES cells by microinjection of ES cells into donor blastocytes to create a chimeric animal, which chimeric animal can be bred to produce an animal in which every cell contains the targeted modification. A transgenic animal can be produced by introducing nucleic acid into the male pronuclei of a fertilized oocyte, e.g., by microinjection, retroviral infection, and allowing the oocyte to develop in a pseudopregnant female foster animal. Further, random transgenic animals containing an exogenous OPSDL3 gene, e.g., a human OPSDL3 gene, may be useful in an in vivo context since various physiological factors that are present in vivo and that could effect ligand binding, OPSDL3 activation, and signal transduction, may not be evident from in vitro cell-free or cell-based assays. Accordingly, it is useful to provide non-human transgenic animals to assay in vivo OPSDL3 protein function, including ligand interaction, the effect of specific mutant OPSDL3 proteins on OPSDL3 protein function and ligand interaction, and the effect of chimeric OPSDL3 proteins. It is also possible to assess the effect of null mutations, that is mutations that substantially or completely eliminate one or more OPSDL3 protein functions.

### SPECIFIC EMBODIMENTS

As described below, wild-type (n=12) and OPSDL3 knock-out (n=10) mice were tested in a hot plate test and a tail flick test. The hot plate test, which is a measure of centrally-mediated pain, did not detect a significant difference between the two groups. The tail flick test, which is a measure of spinally-mediated pain, detected a significant decrease in tail flick latency for the OPSDL3 knock-out mice, indicating that the knock-out animals had a greater reaction to spinal pain than the wild-type group. Further experiments described in Example 4 suggest that activation of OPSDL3 results in an increase in cAMP.

### EXAMPLES

The following example is put forth so as to provide those of ordinary skill in the art with a complete disclosure and description of how to make and use the methods and compositions of the invention, and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

### Example 1

### Expression of Human OPSDL3

Knock-out mice containing a lacZ gene insertion into the endogenous OPSDL3 gene were generated as described in co-pending U.S. Pat. No. 6,586,251, herein specifically incorporated by reference in its entirety. LacZ expression in the knock-out mice was analyzed. The results show that the areas of the spinal cord where OPSDL3 expression is highest correspond to regions of the spinal cord and medulla where unmyelinated (C-fibers) and small diameter myelinated (Ab fibers) primary afferents terminate. These small diameter sensory afferents are known to transmit the sensation(s) of pain from the periphery to the central nervous system. More specifically, OPSDL3 is expressed predominantly by neurons within the gray matter of the spinal cord and are most abundant in the superficial layers of dorsal horn, particularly the marginal zone and substantia gelatinosa (Rexed's laminae 1 and 2), with fewer cells present in the nucleus proprius (lamina III/IV) and around the central canal (lamina X). Cells expressing OPSDL3 are present in these areas throughout the length of the spinal cord, as well as in the medulla within the contiguous, homologous portions of the spinal nucleus of the trigeminal nerve. The topographic distribution of OPSDL3 expressing cells in the spinal cord and medulla corresponds closely to the distribution of mu opioid receptors. The mu subclass of opioid receptors is known to be specifically involved in mediating the analgesic effects of morphine and related opiates, as well as that of endogenous opioid-like peptides (Sora et al. (1997) Proc. Natl. Acad. Sci. 94:1544-1549).

### Example 2

### Hot Plate Test

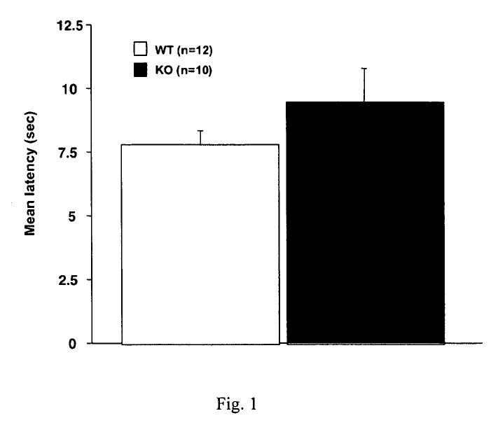

Mice were placed within a Plexiglas cylinder on a 55° C. hot plate. Animals were timed from the time that their feet came in contact with the hot surface until the time when they gave a behavioral response indicative of pain sensation. Specifically, timing was terminated when animals either licked their hindpaws or attempted to escape from the cylinder. Latency to pain-related behavioral response was used as a measure of nociceptive threshold in both wild type and OPSDL3 knock-out mice. A Student's independent groups t-test was used to compare the hot plate latencies for the two groups. The results are shown in FIG. 1. There was no significant difference between OPSDL3 knock-outs and wild type mice in this measure of acute, supraspinal, thermal pain (t(15)=0.242, p>0.81).

### Example 3

### Tail Flick Test

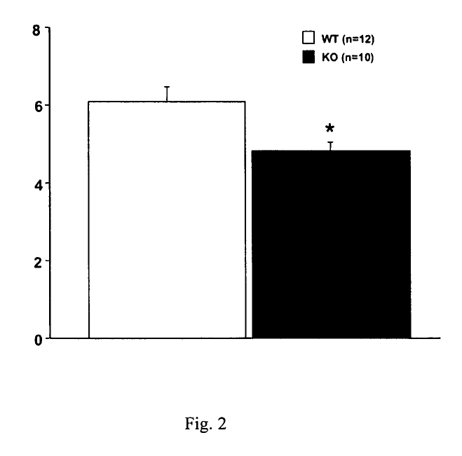

Mice were gently held on a platform of an automated apparatus wrapped in a soft cloth. Their tails were exposed and extended in a straight line along a narrow groove. Once the tail was laying flat in the groove, the experimenter activated a high-intensity and heat producing narrow beam of light that was directed at a small spot in the tail. When the animal reached its pain threshold, a spinal reflex caused the tail to “flick” out of the light beam, automatically stopping a timer that started when the beam was activated. Each animal was tested 3 times, on different regions of the tail, and the median latency to “flick” was recorded as the nociceptive threshold. Experiments were performed blind with respect to the animals' genotype. A Student's independent groups t test was used to compare the nociceptive threshold for the two groups. The results are shown in FIG. 2. OPSDL3 knock-outs were significantly more sensitive to pain than wild types in this acute, thermal, spinal pain assay (t(15)=3.615, p<0.0025).

### Example 4

### Signaling Properties of Human OPSD-like3

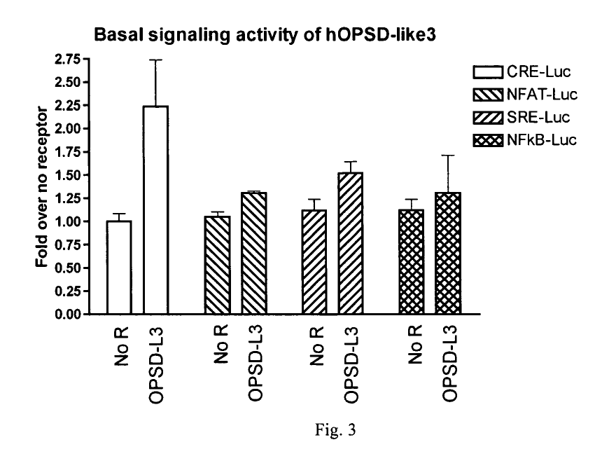

Human OPSD-like3 (hOPSDL3) cDNA was cloned into the expression vector pcDNA3.1-(pcDNA3.1-hOPSDL3). HEK-293 hz cells were transfected with pcDNA3.1-hOPSDL3 along with the luciferase reporter plasmids CRE-luciferase, SRE-luciferase, NFAT-luciferase and NFkB-luciferase. The basal signaling activity of hOPSDL3 was tested with each of these reporters by performing a luciferase activity assay 48 hours after transfection. The results are shown in FIG. 3. Transfection of hOPSD-like3 caused an appreciable increase in the CRE-luciferase activity suggesting that activation of hOPSDL3 leads to an increase in intracellular cyclic-AMP (cAMP).

What is claimed is:

1. A method for identifying an agent capable of modulating an OPSDL3 protein, or protein fragment, comprising:
(a) contacting a test agent with a cell expressing an OPSDL3 protein, or protein fragment; and
(b) determining the ability of the test agent to modulate OPSDL3 protein or protein fragment relative to a control.

2. The method of claim 1, wherein the ability of the test agent to modulate OPSDL3 is determined by a modulation of cAMP level relative to the control.

3. The method of claim 2, wherein an increase in cAMP indicates an agent capable of activating OPSDL3.

4. A method for identifying an agent capable of modulating an OPSDL3 protein, or protein fragment, comprising:
(a) administering a test agent to an animal expressing an OPSDL3 protein, or protein fragment; and
(b) determining the ability of the test agent to modulate OPSDL3 protein or protein fragment, wherein an agent capable of inhibiting or activating OPSDL3 protein activity or expression is a modulator of OPSDL3.

5. The method of claim 4, wherein the ability of the agent to activate OPSDL3 in a test animal is determined by a tail flick assay.

6. The method of claim 4, wherein the agent is an activator or agonist of OPSDL3.

7. A method for alleviating or inhibiting pain in a subject suffering therefrom, comprising administering an effective amount of an agent capable of activating human OPSD-like3 (OPSDL3), wherein the agent is the activator or agonist of claim 6.

9. The method of claim 8, wherein the activator or agonist is peptide, a small molecule, an activating antibody or fragment thereof.

10. A non-human mammal comprising a modification of an endogenous OPSDL3 gene.

11. The non-human mammal of claim 10, wherein the endogenous OPSDL3 gene is altered or deleted such that the function of the endogenous OPSDL3 protein is reduced or ablated.

12. The non-human mammal of claim 11, further comprising a human OPSDL3 transgene.
